(19) United States
(12) Patent Application Publication
(10) Pub. No.: US 2026/0137668 A1
(43) Pub. Date: May 21, 2026

(54) EZH2 INHIBITION THERAPIES FOR THE TREATMENT OF BRCA1-ASSOCIATED PROTEIN (BAP1) MUTATED CANCERS

(71) Applicant: Constellation Pharmaceuticals, Inc., Boston, MA (US)

(72) Inventors: Archana Bommi-Reddy, Milton, MA (US); Patricia Joyce Keller, Shorewood, MN (US); Patrick Trojer, Reading, MA (US)

(21) Appl. No.: 19/118,565

(22) PCT Filed: Oct. 5, 2023
(86) PCT No.: PCT/US2023/034522

§ 371 Date: Apr. 4, 2025

Related U.S. Application Data
(60) Provisional application No. 63/413,647, filed on Oct. 6, 2022.

(51) Int. Cl. A61K 31/443; A61K 9/00; A61P 35/00

(52) U.S. Cl. CPC A61K 31/443; A61K 9/0053; A61P 35/00

(57) ABSTRACT

Provided herein is (R)-7-chloro-2-((1r,4R)-4-(3-methoxyaetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide, or a pharmaceutically acceptable salt thereof for use in methods of treating certain cancers having at least one BAP1 mutation.

### RELATED APPLICATION

This application claims priority to U.S. provisional application No. 63/413,647, filed Oct. 6, 2022, the entire content of which is incorporated herein by reference.

### BACKGROUND

Mutations of the BRCA1-associated protein (BAP1) gene are associated with a substantially increased risk for developing cancers including melanoma, mesothelioma, and renal cell carcinoma. See e.g., Cancer Genetics, (2021) vol. 256-257, 31-35; Cancer Discov (2020) 10 (8): 1103-112 BAP1 loss in mice can result in increased H3K27me3 levels, increased EZH2 expression and enhanced repression of PRC2 targets. See e.g., Nat Med (2015) 21 (11): 1344-1349. In addition, it has recently been shown that cancers such as mesothelioma harboring BAP1 mutations show resistance to traditional platinum-based chemotherapies. See e.g., Clin Cancer Res (2021) 27 (8): 2277-2291. To date, advances towards treating BAP1-mutated cancers have been limited. New approaches to treat cancers harboring one or more BAP1 mutations are therefore needed.

### SUMMARY

It has now been found that mesotheliomas with BAP1 mutations are treatable with the EZH2 inhibitor (R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide, herein referred to as Compound 1. See e.g., the clinical data in Table 1. Therefore, in one aspect, provided herein are methods of using Compound 1 to treat cancers such as mesothelioma having at least one BAP1 mutation. Also provided are uses of Compound 1 for the manufacture of medicaments for treating said cancers. In another aspect, provided is Compound 1 for use in the treatment of cancer, such as mesothelioma, wherein e.g., such cancer harbors at least one BAP1 mutation.

### BRIEF DESCRIPTION OF THE FIGURES

FIG. 1 shows the treatment duration of Compound 1 in subjects with malignant mesothelioma harboring BAP1 loss mutations.

FIG. 2 shows tumor growth inhibition (TGI) by treatment with Compound 1 in BAP1 altered patient derived xenograft (PDX) models of mesothelioma.

FIG. 3A shows mean±SEM absolute tumor volumes of vehicle control and treatment groups of mesothelioma PDX models PXF 537 over time. Control Vehicle: 10 ml/kg/day phosphate buffer po. Treatment with Compound 1: 75 mg/kg/day po.

FIG. 3B shows mean±SEM absolute tumor volumes of vehicle control and treatment groups of mesothelioma PDX models PXF 541 over time. Control Vehicle: 10 ml/kg/day phosphate buffer po. Treatment with Compound 1: 75 mg/kg/day po.

FIG. 3C shows mean±SEM absolute tumor volumes of vehicle control and treatment groups of mesothelioma PDX models PXF 680 over time. Control Vehicle: 10 ml/kg/day phosphate buffer po. Treatment with Compound 1: 75 mg/kg/day po.

FIG. 3D shows mean±SEM absolute tumor volumes of vehicle control and treatment groups of mesothelioma PDX models PXF 1118 over time. Control Vehicle: 10 ml/kg/day phosphate buffer po. Treatment with Compound 1: 75 mg/kg/day po.

FIG. 3E shows mean±SEM absolute tumor volumes of vehicle control and treatment groups of mesothelioma PDX models PXF 2328 over time. Control Vehicle: 10 ml/kg/day phosphate buffer po. Treatment with Compound 1: 75 mg/kg/day po.

FIG. 3F shows mean±SEM absolute tumor volumes of vehicle control and treatment groups of mesothelioma PDX models PXF 2443 over time. Control Vehicle: 10 ml/kg/day phosphate buffer po. Treatment with Compound 1: 75 mg/kg/day po.

### DETAILED DESCRIPTION

In a first embodiment, provided are methods of treating a cancer in a subject comprising administering to the subject an effective amount of Compound 1, or a pharmaceutically acceptable salt thereof, wherein the cancer has at least one BAP1 mutation. Also provided as part of a first embodiment are uses of an effective amount of Compound 1, or a pharmaceutically acceptable salt thereof, for the manufacture of a medicament for treating a cancer having at least one BAP1 mutation. Also provided as part of a first embodiment are uses of an effective amount of Compound 1, or a pharmaceutically acceptable salt thereof, for treating a cancer having at least one BAP1 mutation. Also provided as part of a first embodiment, are pharmaceutical compositions comprising an effective amount of Compound 1, or a pharmaceutically acceptable salt thereof, for treating a cancer having at least one BAP1 mutation.

Compound 1 and (R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide are used interchangeably and each refer to the compound having the following chemical structure.

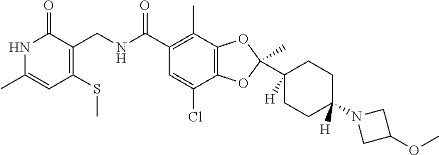

In one aspect, as part of a second embodiment, the cancer treated by the present methods is selected from a melanoma, mesothelioma, renal cell carcinoma, thymic cancer, salivary gland cancer, and cholangiocarcinoma. In another aspect, as part of a second embodiment, the cancer treated by the present methods is mesothelioma. In another aspect, as part of a second embodiment, the cancer treated by the present methods is malignant mesothelioma. In another aspect, as part of a second embodiment, the cancer treated by the present methods is pleural or peritoneal malignant mesothelioma. In another aspect, as part of a second embodiment, the cancer treated by the present methods is refractory or relapsed malignant mesothelioma. In another aspect, as part of a second embodiment, the cancer treated by the present methods is refractory or relapsed pleural or peritoneal malignant mesothelioma. In another aspect, as part of a second embodiment, the cancer treated by the present methods is melanoma. In another aspect, as part of a second embodiment, the cancer treated by the present methods is uveal melanoma.

In a third embodiment, the at least one BAP1 mutation of the present methods (e.g., as in the first or second embodiment) is a loss of function (LOF) mutation. Alternatively, as part of a third embodiment, the at least one BAP1 mutation of the present methods (e.g., as in the first or second embodiment) results in a loss of BAP1 expression. In a third embodiment, the at least one BAP1 mutation of the present methods (e.g., as in the first or second embodiment) is characterized as a frameshift, missense, nonsense, insertion, deletion, splice acceptor variant, or stop gained mutation. In one aspect the at least one BAP1 mutation is a p.G194R mutation or p.L49Qfs*18 mutation. In one aspect the at least one BAP1 mutation is in the catalytic domain of BAP1.

As used herein, an BAP1 LOF mutation refers to a mutation which reduces or abolishes BAP1 protein function. LOF may be due by loss of expression due to nonsense mediated decay loss of activity or due to truncation of the protein (missing critical residues or domains).

“Relapsed” mesothelioma or a subject who is characterized as relapsed means that the subject having the cancer at one time responded to a given treatment for the cancer (e.g., treatment with a platinum therapy such as cisplatin), but who no longer responds. “Refractory” mesothelioma or a subject who is characterized as refractory means that the subject having the cancer is unresponsive or demonstrates worsening of disease while on a given treatment (e.g., while being treated with a platinum therapy such as cisplatin).

The terms “treatment,” “treat,” and “treating” refer to reversing, alleviating, delaying the onset of, or inhibiting the progress of a cancer or one or more symptoms of a disease described herein. In some embodiments, treatment may be administered after one or more signs or symptoms of a cancer have developed or have been observed (i.e., therapeutic treatment). In other embodiments, treatment may be administered in the absence of signs or symptoms of a cancer. For example, treatment may be administered to a susceptible subject prior to the onset of symptoms (i.e., prophylactic treatment) (e.g., in light of a history of symptoms and/or in light of an exposure to a pathogen). In further embodiments, treatment includes delaying the onset of at least one symptom of a cancer for a period of time. Treatment may also be continued after symptoms have resolved, for example, to delay or prevent recurrence (i.e., maintenance treatment).

The terms “subject” and “patient” may be used interchangeably, and mean a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “effective amount” or “therapeutically effective amount” refers to an amount of Compound 1, or a pharmaceutically acceptable salt thereof, that will elicit a biological or medical response of a subject e.g., a dosage of between 0.01-100 mg/kg body weight/day. In one aspect, as part of a fourth embodiment, the effective amount of Compound 1 in the present methods (e.g., in any one of the first to third embodiments) ranges from about 10 mg/kg body weight/day to about 150 mg/kg body weight/day. In another aspect, as part of a fourth embodiment, the effective amount of Compound 1 in the present methods (e.g., in any one of the first to third embodiments) ranges from about 50 mg to about 375 mg daily. In another aspect, as part of a fourth embodiment, the effective amount of Compound 1 in the present methods (e.g., in any one of the first to third embodiments) ranges from about 200 mg to about 400 mg daily. In one aspect, as part of a fourth embodiment, the effective amount of Compound 1 in the present methods (e.g., in any one of the first to third embodiments) ranges from about 200 mg to about 300 mg daily. In another aspect, as part of a fourth embodiment, the effective amount of Compound 1 in the present methods (e.g., in any one of the first to third embodiments) is about 250 mg daily. In another aspect, as part of a fourth embodiment, the effective amount of Compound 1 in the present methods (e.g., in any one of the first to third embodiments) ranges from about 325 mg to about 400 mg daily. In another aspect, as part of a fourth embodiment, the effective amount of Compound 1 in the present methods (e.g., in any one of the first to third embodiments) ranges from about 325 mg to about 375 mg daily. In another aspect, as part of a fourth embodiment, the effective amount of Compound 1 in the present methods (e.g., in any one of the first to third embodiments) is about 350 mg daily. In another aspect, as part of a fourth embodiment, the effective amount of Compound 1 in the present methods (e.g., in any one of the first to third embodiments) is about 375 mg daily. In one aspect, as part of a fourth embodiment, the effective amount of a pharmaceutically acceptable salt of Compound 1 in the present methods (e.g., in any one of the first to third embodiments) is equivalent to an amount of Compound 1 ranging from about 10 mg/kg body weight/day to about 150 mg/kg body weight/day. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 50 mg to about 375 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 200 mg to about 400 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 200 mg to about 300 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 is about 250 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 325 mg to about 400 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 325 mg to about 375 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 of about 350 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 is about 375 mg daily.

Methods of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Sterile injectable forms of Compound 1 described herein may be aqueous or oleaginous suspension. These suspensions may be formulated according to techniques known in the art using suitable dispersing or wetting agents and suspending agents. In one aspect, as part of a fifth embodiment, Compound 1 in the present methods (e.g., in any one of the first to fourth embodiments) is administered orally.

Compound 1 may be present in the form of a pharmaceutically acceptable salt. For use in medicines, pharmaceutically acceptable salt refers to non-toxic “pharmaceutically acceptable salts.” Pharmaceutically acceptable salt forms include pharmaceutically acceptable acidic/anionic or basic/cationic salts where possible.

Compound 1, or a pharmaceutically acceptable salt thereof, may be formulated as part of a pharmaceutical composition comprising Compound 1, or a pharmaceutically acceptable salt thereof, together with a pharmaceutically acceptable carrier. Pharmaceutically acceptable carriers, (e.g. carriers, adjuvants or vehicles) that may be used in the compositions described herein include, but are not limited to, ion exchangers, alumina, aluminum stearate, lecithin, serum proteins (such as human serum albumin), buffer substances (such as phosphates), glycine, sorbic acid, potassium sorbate, partial glyceride mixtures of saturated vegetable fatty acids, water, salts or electrolytes (such as protamine sulfate, disodium hydrogen phosphate, potassium hydrogen phosphate, sodium chloride, zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “pharmaceutically acceptable carrier” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

In one aspect, as part of a sixth embodiment, Compound 1 in the disclosed methods (e.g., in any one of the first to fifth embodiments) is present in a crystalline form. Crystalline forms of Compound 1 are disclosed in WO 2021/016414 and are incorporated by reference herein. In another aspect, as part of a sixth embodiment, Compound 1 in the disclosed methods (e.g., in any one of the first to fifth embodiments) is of crystalline Form 1 characterized by at least three X-ray powder diffraction peaks at 2Θ angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least four X-ray powder diffraction peaks at 2Θ angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least five X-ray powder diffraction peaks at 2Θ angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least six X-ray powder diffraction peaks at 2Θ angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by X-ray powder diffraction peaks at 2Θ angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by X-ray powder diffraction peaks at 2Θ angles selected from 10.0°, 10.2°, 12.3°, 12.7°, 13.3°, 14.9°, 15.3°, 20.2°, 20.8°, 21.3°, 22.2°, 22.5°, and 23.8°. In another aspect, as part of a sixth embodiment, Compound 1 in the disclosed methods (e.g., in any one of the first to fifth embodiments) is of crystalline Form 1 characterized by X-ray powder diffraction peaks at 2Θ angles selected from 10.0°, 10.2°, 11.00, 11.40, 11.80, 12.30, 12.70, 13.30, 14.90, 15.30, 16.10, 17.40, 20.20, 20.80, 21.30, 22.20, 22.5°, and 23.8°. In another aspect, as part of a sixth embodiment, Compound 1 in the disclosed methods (e.g., in any one of the first to fifth embodiments) is of crystalline Form 1 characterized by x-ray powder diffraction peaks at 2Θ angles selected from 14.9°, 20.2°, and 20.8°. In another aspect, as part of a sixth embodiment, Compound 1 in the disclosed methods (e.g., in any one of the first to fifth embodiments) is of crystalline Form 1 characterized by x-ray powder diffraction peaks at 2Θ angles selected from 10.0°, 14.9°, 20.2°, and 20.8°. In another aspect, as part of a sixth embodiment, Compound 1 in the disclosed methods (e.g., in any one of the first to fifth embodiments) is of crystalline Form 1 characterized by x-ray powder diffraction peaks at 2Θ angles selected from 10.0°, 14.9°, 20.2°, 20.8°, and 22.2°. In another aspect, as part of a sixth embodiment, Compound 1 in the disclosed methods (e.g., in any one of the first to fifth embodiments) is of crystalline Form 1 characterized by x-ray powder diffraction peaks at 2Θ angles selected from 10.0°, 13.3°, 14.9°, 20.2°, 20.8°, and 22.2°.

In one aspect, as part of a seventh embodiment, Compound 1 in the disclosed methods (e.g., in any one of the first to fifth embodiments), or a pharmaceutically acceptable salt thereof, is present as a solid dispersion comprising amorphous (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide or a pharmaceutically acceptable salt thereof; and a pharmaceutically acceptable polymer. In some aspects, the pharmaceutically acceptable polymer is selected from polyvinylpyrrolidone (PVP), polyvinylpyrrolidone/vinyl acetate copolymer (PVP-VA), hydroxypropyl methylcellulose (HPMC), hypromellose phthalate (HPMC-P), and hypromellose acetate succinate (HPMC-AS), preferably HPMC or HPMC-AS, more preferably HPMC-AS grade M. In some aspects, the weight ratio of the pharmaceutically acceptable polymer to (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide ranges from 10:90 wt % to 90:10 wt %, from 15:85 wt % to 85:15 wt %, from 20:80 wt % to 80:20 wt %, from 25:75 wt % to 75:25 wt %, from 30:70 wt % to 70:30 wt %, from 35:65 wt % to 65:35 wt %, from 40:60 wt % to 60:40 wt %, or from 45:55 wt % to 55:45 wt %, preferably from 25:75 wt % to 75:25 wt %, from 30:70 wt % to 70:30 wt %, from 40:60 wt % to 60:40 wt %, or from 45:55 wt % to 55:45 wt %, more preferably from 20 wt % to 40 wt % or from 25 wt % to 35 wt %, or is 50%. Other aspects of the solid dispersion are described in WO 2018/136596.

In one aspect, as part of an eighth embodiment, Compound 1 in the disclosed methods (e.g., in any one of the first to seventh embodiments) or a pharmaceutically acceptable salt thereof, is administered for a period of at least about 4 days, at least about 6 days, at least about 8 days, at least about 12 days, at least about 18 days, at least about 30 days, at least about 60 days, at least about 6 months, or at least about 1 year.

### EXEMPLIFICATION

### Preparation of Compound 1

(R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide was prepared following the procedures described in PCT/US2019/027932 and PCT/US2020/043163, each of which are incorporated by reference herein.

### Example 1

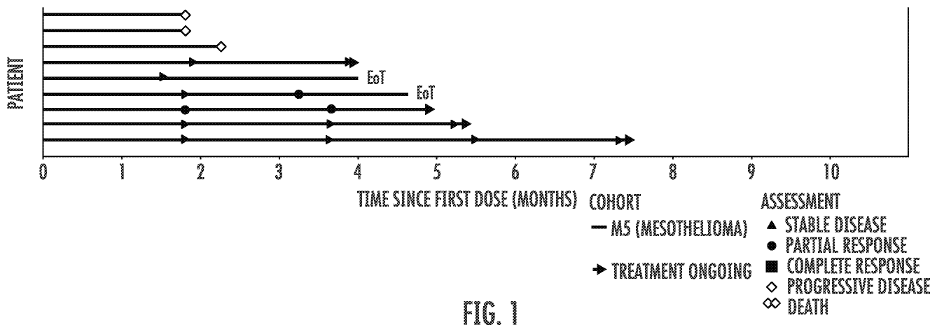

As part of a human clinical phase 2 study, subjects with pleural or peritoneal BAP1 loss mesothelioma were administered 350 mg/day Compound 1 for up to about 7.5 months (FIG. 1). Compound 1 was administered orally as a single agent (monotherapy). The cancers in each subject were known to have BAP1 mutations as determined by next generation sequencing (NGS) or local immunohistochemistry (IHC) testing prior to treatment. The response by cancer cohort for efficacy evaluable patients at an intermediate cut-off date is shown in Table 1. A complete response is characterized as disappearance of all lesions, a partial response is characterized as at least a 30% decrease in the sum of the longest diameter (LD) of target lesions, taking as reference the baseline sum LD; stable disease is characterized as neither sufficient shrinkage to qualify for partial response nor sufficient increase to qualify for progressive disease, taking as reference the smallest sum diameters; and progressive disease is characterized as at least a 20% increase in the sum of the LD of target lesions, taking as reference the smallest sum LD recorded since the treatment started or the appearance of one or more new lesions.

TABLE 1

| Category, n (%) | (N = 9) |
|---|---|
| Complete response | 0 |
| Partial response | 2 (22.2) |
| Stable disease | 4 (44.4) |
| Progressive disease | 3 (33.3) |

### Example 2

The ability of Compound 1 to selectivity suppress mesothelioma cancer cell viability was assessed in a panel of cell lines after 7, 14 and 21 days of treatment with Compound 1. Cell viability was determined using CellTiter-Glo®2 luminescent cell viability assay.

Cell culture conditions. MSTO-211H, H2052, H2452, H28, H226 cell lines were obtained from ATCC (Manassas, VA) and were grown in media as recommended by the vendor. For long-term assays, cells were plated in 96-well plates containing. Compound 1. Cell lines were treated with a dose titration of Compound 1 starting at 2 μM top concentration.

Cellular assay—assessment of cell viability. Relative cell numbers were assessed by CellTiter-Glo® 2.0 luminescent cell viability assay (Promega Catalog #G9243) using a Perkin Elmer EnVision Alpha Reader (Model #2104, Waltham, MA). 50 μL of CellTiter-Glo® 2.0 reagent was added to each well containing 100 μL cell suspension and incubated for 30 min at RT with shaking at low speed. GraphPad Prism was used for curve fitting and GI50 determinations.

Phenotypic response to Compound 1 in mesothelioma cell lines. In the mesothelioma cell line panel, after 7 days of treatment, most mesothelioma cell lines show little to no viability effects from treatment with Compound 1. However, prolonged treatment for 14 and 21 days substantially increased the sensitivity of the cell lines. The observed GI50s are summarized in Table 2. At 21 days, 2/3 models that harbor a BAP1 mutation or loss were sensitive to Compound 1 treatment, while 2/2 with intact BAP1 retained unresponsive (Table 2) in this setting. In conclusion, Compound 1 potently affected the cell viability of mutant BAP1 mesothelioma cells in long-term growth assays, with an enrichment of response in BAP1 mutant models. These data suggest, the applicability of Compound 1 in the treatment of BAP1 mutant mesothelioma tumors.

TABLE 2
GI50 values for mesothelioma cells treated
with Compound 1 for 7, 14 and 21 days

| | | GI50 (μM) | | |
|---|---|---|---|---|
| Cell Line | BAP1 status | Day 7 | Day 14 | Day 21 |
| MSTO-211H | wildtype | >2 | >2 | >2 |
| H2052 | wildtype | >2 | >2 | >2 |
| H2452 | mutant | >2 | 0.006 | 0.005 |
| H28 | mutant | >2 | 0.234 | 0.045 |
| H226 | mutant | >2 | >2 | >2 |

### Example 3

Compound 1 was evaluated in 6 patient-derived xenograft (PDX) models of pleural mesothelioma with BAP1 alterations (Table 3) implanted subcutaneously in immunodeficient mice (NSG mice in the case of tumor model PXF 2328, NMRI nu/nu mice for all other tumor models). Each experiment had two groups of generally five mice each enrolled at tumor volumes in the range of 53.9-198.9 mm³. The first group was a vehicle control group, while the second group was treated with Compound 1, administered orally (p.o.) once daily (QD) at a dose level of 75 mg/kg. The duration of the experiments ranged between 30 and 65 days.

TABLE 3
BAP1 alteration characteristics of PDX models used

| Model | BAP1 Alteration | Zygosity |
|---|---|---|
| PXF_537 | missense_variant | homozygous |
| PXF_541 | frameshift_variant | heterozygous |
| PXF_680 | splice_acceptor_variant | heterozygous |
| PXF_1118 | frameshift_variant | homozygous |
| PXF_2328 | stop-gained, splice | homozygous |
| | region_variant | |
| PXF_2443 | frameshift_variant | heterozygous |

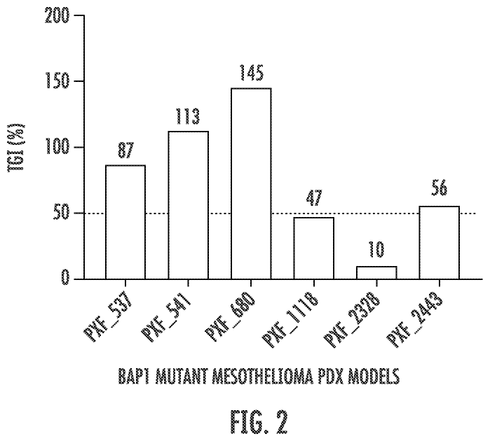

Antitumor efficacy of all groups was assessed using the vehicle control group as a reference. Tumor growth inhibition was determined by the comparison of tumor growth in the test groups with the vehicle control group and is expressed as the maximum tumor growth inhibition (TGI) value in percent. The statistical significance of differences between test and control groups was assessed using the non-parametric U-test. A relevant TGI of greater than 50% was observed in 4 out of the 6 PDX models (FIG. 2).

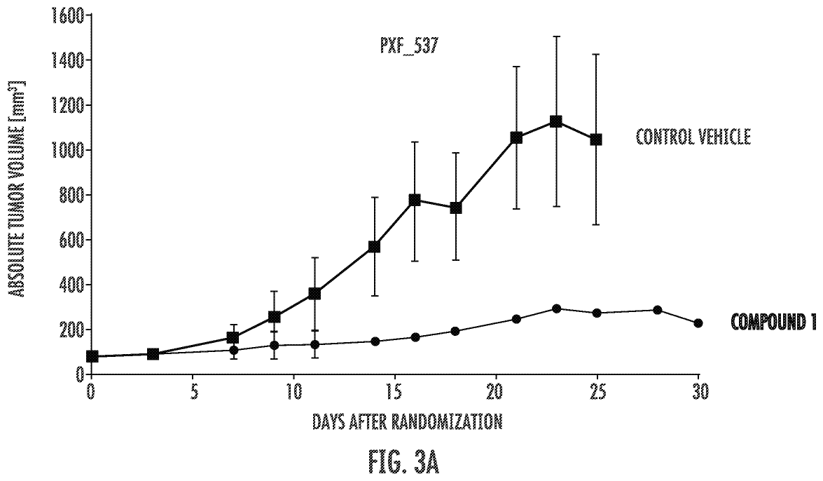

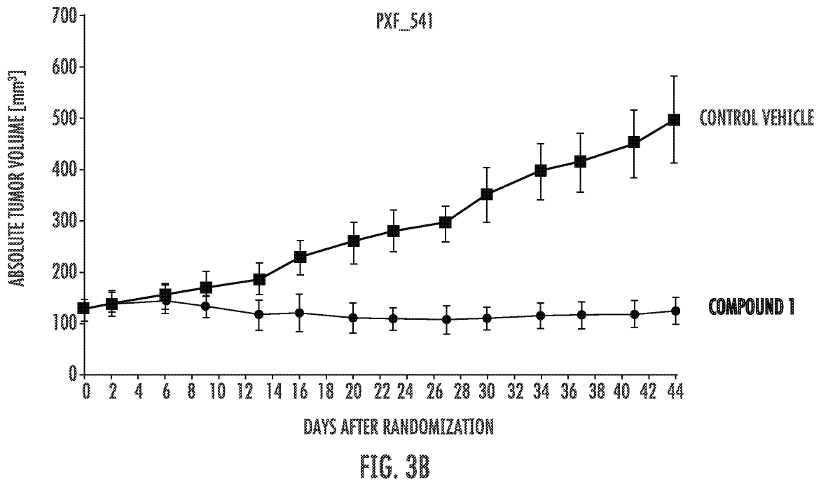

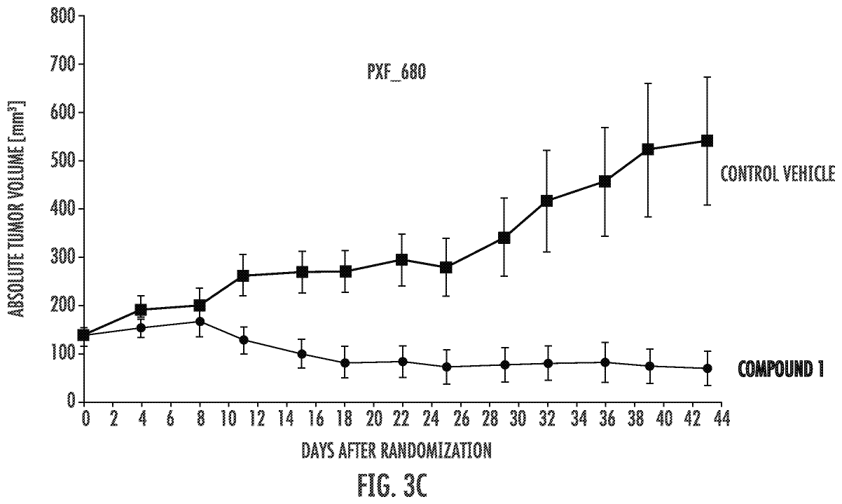

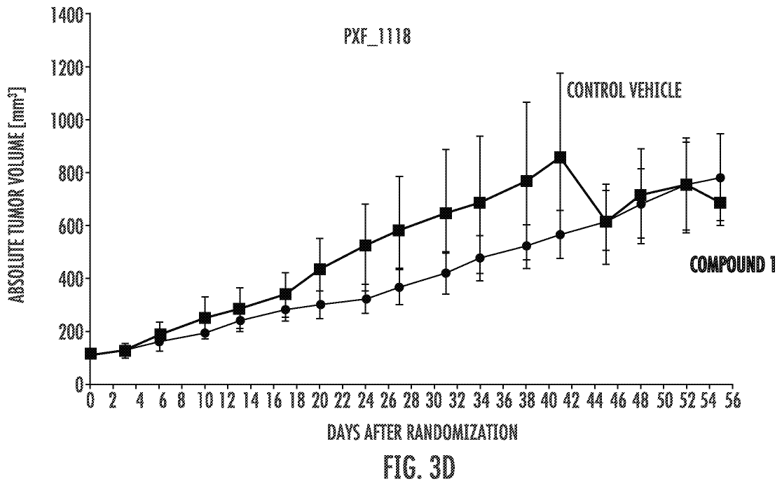

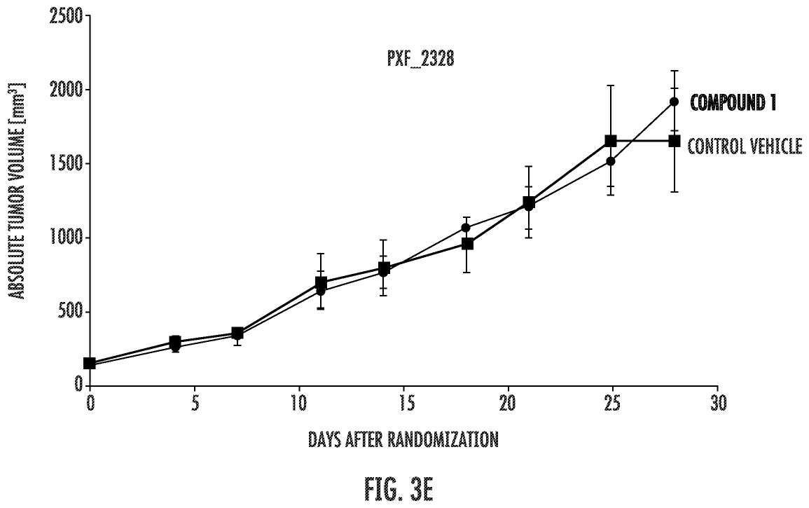

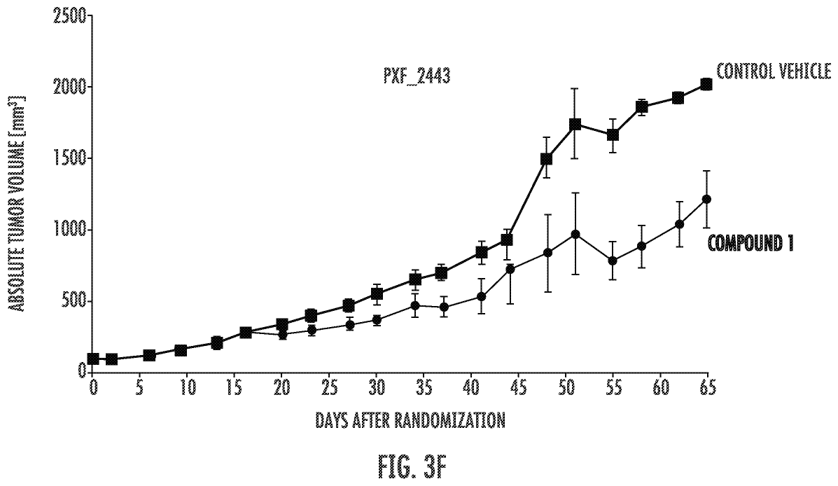

Compound 1 inhibited growth of pleural mesothelioma models PXF 541 and PXF 680 with TGI values>100% and a statistically significant reduction in tumor volume compared to the vehicle control group (non-parametric U-test). Compound 1 inhibited growth of PXF 537 to almost stasis and a statistically significant reduction in tumor volume compared to the control group. Furthermore, Compound 1 slowed growth of PXF 1118 and PXF 2443 without a statistically significant reduction in tumor volume while it was inactive against PXF 2328 implanted in NSG mice (FIG. 3 A-F).

In conclusion, Compound 1 inhibited, reduced and slowed tumor growth in the majority of pleural mesothelioma PDX models. These findings translate well to the clinical setting, where the majority (>60%) of patients showed (partial) response and stable disease at an intermediate cut-off date (Table 1).

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art

1. A method of treating malignant mesothelioma in a subject comprising administering to the subject an effective amount of a compound having the formula: 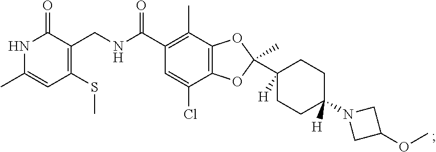
or a pharmaceutically acceptable salt thereof, wherein the malignant mesothelioma has at least one BAP1 mutation.

2. The method of claim 1, wherein the at least one BAP1 mutation is a loss of function (LOF) mutation

3. The method of claim 1 or 2, wherein the at least one BAP1 mutation results in a loss of BAP1 expression.

4. The method of any one of claims 1 to 3, wherein malignant mesothelioma is pleural or peritoneal malignant mesothelioma.

5. The method of any one of claims 1 to 4, wherein malignant mesothelioma is pleural malignant mesothelioma.

6. The method of any one of claims 1 to 4, wherein malignant mesothelioma is peritoneal malignant mesothelioma.

7. The method of any one of claims 1 to 6, wherein malignant mesothelioma is characterized as relapsed or refractory.

8. The method of any one of claims 1 to 7, wherein malignant mesothelioma is characterized as relapsed.

9. The method of any one of claims 1 to 7, wherein malignant mesothelioma is characterized as refractory.

10. The method of any one of claims 1 to 9, wherein the subject is administered about 200 to 400 mg/day of the compound.

11. The method of claim 10, wherein the subject is administered about 350 mg/day of the compound.
